(19) United States
(12) Patent Application Publication
(10) Pub. No.: US 2009/0264935 A1
(43) Pub. Date: Oct. 22, 2009

(54) Surgical Implant Used in Osteotomy

(76) Inventor: Pierre Imbert, Fréjus (FR)

Correspondence Address: JAMES C. LYDON, 100 DAINGERFIELD ROAD, SUITE 100, ALEXANDRIA, VA, 22314, US

(21) Appl. No.: 12/084,247

(22) PCT Filed: Oct. 24, 2006
(86) PCT No.: PCT/FR2006/002377

§ 371 Date: Apr. 28, 2008

(30) Foreign Application Priority Data
Oct. 26, 2005 (FR) 05/10912

(51) Int. Cl. A61B 17/80

(52) U.S. Cl. 606/286; 606/280

(57) ABSTRACT

A surgical implant used for stabilizing an opening site in a bone between two osseous corticals (20, 22) having a plate (14) with an upper concave part (16) and a lower concave part (18) in which the osseous cortical parts come to rest against one another. The concave parts are V-shaped grooves, each pressing against osseous corticals whereby canceling out the forces that tend to expel the plate to the front as well as the forces that tend to cause the plate to enter the osseous opening.

### TECHNICAL FIELD

This invention relate to surgical implants used in osteotomy in order to stabilize openings made in bones, and relates in particular to a surgical implant used in osteotomy.

### STATE OF THE ART

The surgical intervention referred to as osteotomy is practiced with the intention of modifying the axis of a long bone, wherein the intention is to correct a congenital or acquired deformation, or in modifying the axis of the bone, to alter the distribution of loads applied to the overlying or underlying joint surfaces.

The most current example is that of tibial valgus osteotomy by internal addition. The purpose of this intervention, by outwardly angling the axis of the tibia, is to decrease the forces applied to the internal compartment of the knee, which is the most frequently used, and to increase those to which the outer compartment is subjected, in general protected from wear.

Currently, in order to affix the site of the osteotomy until the osseous consolidation takes place, a simple plate is screwed onto the bone, which presents the drawback of transferring the loads on the individual screws with the risk that the screws will migrate or rupture. This technique is improved by using screw heads that can lock into the plate, together forming a mono-block assembly with a better mechanical resistance. But the support forces are always shared by the individual screws.

A screwed plate can also be utilized comprising a wedge at its central part, which is inserted into the osteotomy site. Unfortunately, its support is principally on the spongy part and there is always a risk of expulsion of the plate by the expulsion forces generated by the vertical compression.

The documents U.S. 2003/125740 and U.S. 2002/120335 disclose a technique closely related to the osteotomy called laminoplasty. This consists of releasing the clearing out the spinal marrow in the osseous tube (the vertebral column) that has become underdeveloped due to the development of arthritis and where the bone is cut along its length, plates are put transversally in place in a space created in order to prevent the tube from reclosing. Each plate has two concave parts against which the two parts of the bone that result from the cutting of the bone are kept supported. But this type of plate is meant to remain in place, contrary to osteotomy, and it cannot be utilized in osteotomy insofar as, in this technique, only the cortical parts of the bone are required to come to rest on the plate by preventing the latter from being able to be expelled or from entering into the osseous opening.

### DISCLOSURE OF THE INVENTION

The purpose of the invention is therefore to realize a surgical implant used for the site of osteotomy that does not risk being expelled or from entering into the osseous opening.

The object of the invention is therefore a surgical implant meant to stabilize opening site in a bone between two osseous corticals, comprising a plate having an upper concave part and a lower concave part in which the osseous cortical parts come to rest against one another. The concave parts are V-shaped grooves adapted to serve as support respectively to the osseous corticals so as to neutralize the forces tending to expel the plate to the front as well as the forces tending to cause the plate to enter the osseous opening.

### BRIEF DESCRIPTION OF THE FIGURES

The purposes, objectives and characteristics of the invention will appear more clearly with the reading of the following description, made in reference to the drawings, in which:

FIG. 1 represents a cross-section of the implant in place in the osteotomy site.

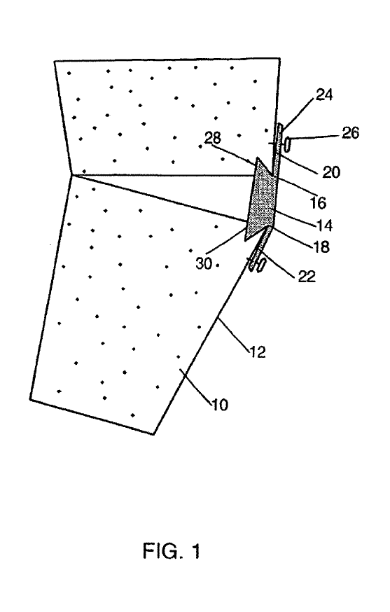

FIG. 2 represents a front view of the implant in place.

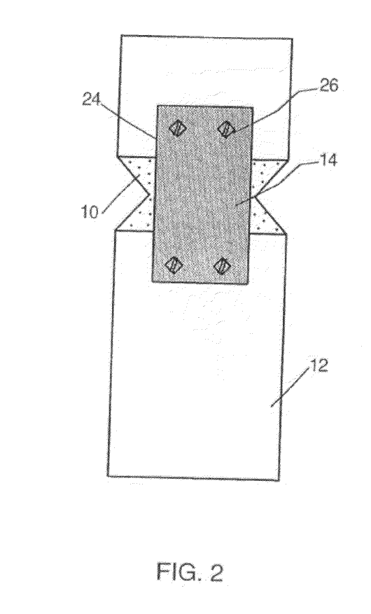

### DETAILED DESCRIPTION OF THE INVENTION

The invention is based on the fact that the bones are constituted of a spongy bone 10 at the center, having a poor mechanical resistance that is surrounded at its periphery by the very dense and very resistant cortical part 12.

In reference to the figures, the implant is constituted by a plate 14 of a thickness and height adapted to the anatomical site, comprising two concave parts in the form of an upper groove 16 and a lower groove 18, each one V-shaped, in which the osseous corticals 20 and 22 are respectively supported.

Because each groove 16 and 18 is V-shaped and therefore has a bottom forming an acute angle, cortical 20 or 22 is blocked, which prevents the plate from being able to be displaced transversally with respect to the corticals. Consequently, this tight support allows the neutralizing of forces tending to expel the plate to the front as well as the forces tending to cause the plate to enter the osseous opening.

On both sides of this central area, the plate can be extended on both sides by a thinner part, like the portion of plate 24 allowing the increasing of stability of the assembly by screwing into the cortical bone by means of short screws such as screw 26. Note that the use of screws can be dispensed with because of the grooves 16 and 18 according to the invention.

In order to further better stabilize the plate, the interior part of the plate preferably has an upper extension 28 and a lower extension 30 that come to be inserted in the spongy part 10 of the bone. Thus, this insertion comes to add to the blockage of the cortical parts of the bone in the V-grooves in order to keep the plate from being displaced.

It is noted that, having a variable size comprising between 3 and 20 mm measured between the bottom of the upper groove and the bottom of the lower groove, the implant according to the invention can be adapted to all the heights of openings and all the bones of the skeleton that could require this type of surgical intervention.

The purpose of the following description is to better understand the invention with regard to the surgical technique of tibial valgus osteotomy by internal addition, which is a non-limiting example of an application of the invention.

The invention consists of making an oblique cutaneous incision from top to bottom and from back to front on the internal side of the upper extremity of the tibia. The bone is exposed and, under radiological control, a sawing line is realized with the oscillating saw. It is slightly ascendant from inside to outside and not starting on the external osseous cortical that acts as a hinge. Very gradually, in order to not break this hinge, which uses the plastic deformation capacities of the bone, a wedge is driven into the saw line. Gradually, it allows arriving at the desired opening height to which the height of the lower and upper grooves of the plate must be added. This is then positioned in the opening and the wedge is removed, allowing the corticals to take support in the grooves, the intra-osseous edges of which are impacted in the spongy bone. The assembly, which has already been made stable, can then be completed by the screwing of the extensions of the plate.

It is noted that after the osseous consolidation has been made, that is to say that the bone has grown into the opening that has been made, the plate can be removed.

The advantages of this invention are as follows:
An excellent primary stability by simple insertion of the osseous corticals into the grooves of the implant.
A possible reinforcement of the assembly by the addition of screws.
Its small size, which makes the incision necessary for its implantation shorter and therefore the osteotomy intervention less invasive and faster.
Not having a wedge interposing between the surfaces of the spongy bone, the osseous rehabilitation of the opening site is more complete and the loss of substance less important in the event of an ablation of the plate.
The good stability of the cortical support favors a rapid reperfusion and recovery of the patient.
Because of its small size, the implant makes a potential subsequent implantation of an intra-osseous material possible without having to surgically remove the plate. For example, in the case of the utilization of the invention for a tibial osteotomy, the placement of a prosthesis for the knee subsequent or concomitant with the osteotomy is also facilitated.

1-4. (canceled)

5. A surgical implant intended to stabilize an opening site in a bone between two osseous corticals, said implant comprising
a plate having an upper concave part and a lower concave part in which said osseous cortical parts are respectively supported; wherein said concave parts are V-shaped grooves adapted to serve as support respectively to said osseous corticals so as to neutralize the forces tending to expel said plate to the front as well as the forces tending to cause said plate to enter the osseous opening.

6. The surgical implant of claim 5, wherein said plate is extended at its upper part and at its lower part by a thinner area provided with holes for the passage of screws allowing an increasing of its stability.

7. The surgical implant of claim 6, wherein the interior part of said plate has an upper extension and a lower extension that are adapted to be inserted into a spongy part of bone.

8. The surgical implant of claims 5, having a variable size, comprising between 3 and 20 mm measured between the bottom of the upper groove and the bottom of the lower groove so as to adapt to all the heights of openings and to all the bones of the skeleton that may require this type of surgical intervention.

9. The surgical implant of claim 6, having a variable size, comprising between 3 and 20 mm measured between the bottom of the upper groove and the bottom of the lower groove so as to adapt to all the heights of openings and to all the bones of the skeleton that may require this type of surgical intervention.

10. The surgical implant of claim 7, having a variable size, comprising between 3 and 20 mm measured between the bottom of the upper groove and the bottom of the lower groove so as to adapt to all the heights of openings and to all the bones of the skeleton that may require this type of surgical intervention.
